SEQUENCE LISTING (3 sequences)

SEQ ID NO: 1 - DNA, 64 bp, Artificial Sequence
  synthetic primer miRmp230a for RNAi designer
tgctgaatagcgtcggctttgtcacggttttggccactgactgaccgtgacaaccgacgctatt

SEQ ID NO: 2 - DNA, 64 bp, Artificial Sequence
  synthetic primer miRmp230b for RNAi designer
tgctgaattgttacactgtccttggtgttttggccactgactgacaccaaggagtgtaacaatt

SEQ ID NO: 3 - DNA, 64 bp, Artificial Sequence
  synthetic primer miRmGCC185 for RNAi designer
tgctgtaagatggccgtttctttgctgttttggccactgactgacagcaaagacggccatctta

(19) United States
(12) Patent Application Publication
(10) Pub. No.: US 2011/0030073 A1
(43) Pub. Date: Feb. 3, 2011

(54) THERAPEUTIC TARGETS AND MEDICAMENTS INVOLVING P230/GOLGIN-245

(76) Inventors: Paul Antony Gleeson, Kew (AU); Jennifer Lea Stow, Kenmore Hills (AU); Zi Zhao Lieu, North Melboune (AU); John George Lock, Stockholm (SE); Luke Alexander Hammond, West End (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/745,536

(22) PCT Filed: Nov. 28, 2008
(86) PCT No.: PCT/AU08/01763

§ 371 Date: Oct. 12, 2010

(30) Foreign Application Priority Data
Nov. 30, 2007 (AU) 2007906558

(51) Int. Cl. A61K 39/395; C12N 5/071; C07K 16/00; A01K 67/027; A61P 29/00

(52) U.S. Cl. 800/9; 435/375; 530/389.1; 424/172.1

(57) ABSTRACT

The present invention relates generally to the field of cell biology and in particular the cellular processes surrounding inflammation. Even more particularly, the present invention provides targets for medicaments useful in reducing levels of TNF-alpha, an extracellular pro-inflammatory mediator. The medicaments are therefore useful in modulating inflammatory responses. Model inflammatory disease systems also form part of the present invention.

### FILING DATA

This application is associated with and claims priority from Australian Provisional Patent Application No. 2007906558, filed on 30 Nov. 2007, the entire contents of which, are incorporated herein by reference.

### FIELD

The present invention relates generally to the field of cell biology and in particular the cellular processes surrounding inflammation. Even more particularly, the present invention provides targets for medicaments useful in reducing levels of an extracellular pro-inflammatory mediator. The medicaments are therefore useful in ameliorating the effects on an inflammatory response. Model inflammatory disease systems also form part of the present invention.

### BACKGROUND

Bibliographic details of the publications referred to by the author in this specification are collected at the end of the description.

Reference to any prior art in this specification is not, and should not be taken as, an acknowledgment or any form of suggestion that this prior art forms part of the common general knowledge in any country.

Tumor Necrosis Factor alpha (TNFα) is the main pro-inflammatory cytokine made and secreted by inflammatory macrophages. Early release of TNFα in response to lipopolysaccharide (LPS) or other inflammatory signals works to activate and recruit T cells and ensures robust innate and acquired immune responses. The excessive secretion of TNFα is also a prevalent and clinically significant problem in acute inflammation and in chronic inflammatory disease. Anti-TNFα treatments have shown success in the treatment of rheumatoid arthritis, inflammatory bowel disease and other conditions. Now improved anti-TNFα strategies that can offer more constrained or cell type specific control of TNFα secretion are being sought. This requires identification of molecular mediators of TNFα secretion, particularly those that can be targeted to block TNFα release.

The export of TNFα requires a secretory pathway whereby the transmembrane precursor of TNFα is transported from the trans-Golgi network (TGN) in tubular carriers that fuse with the recycling endosome (RE) as an intermediate compartment. The RE contributes membrane for the formation of phagocytic cups during ingestion of microbes or particles in these phagocytic cells. TNFα, but not other cytokines, is delivered to the phagocytic cup along with the RE as a means of surface delivery for TACE-mediated cleavage and release. While the latter stages of TNFα secretion via this pathway are beginning to come to light, an earlier but critical phase of TNFα transport out of the TGN is not yet understood.

One class of components which regulates membrane transport from the TGN is the golgins; long coiled coil proteins which are specifically recruited to the subdomains and tubules which emerge from the TGN. There are four human TGN golgins, namely p230/golgin-245, golgin-97, GCC185 and GCC88. These golgins are peripheral membrane proteins that have a TGN targeting sequence located at the C-terminus, called the GRIP domain. Recruitment of p230/golgin-245 and golgin-97 to the TGN is mediated through an interaction with the small G protein, Arl1.

Although both p230 and golgin-97 are effectors of Arl1, the two golgins are localized to distinct membrane domains of the TGN. Distinct spatial segregation of p230 and golgin-97 is also reflected in their function. Golgin-97, but not p230, is associated with distinct membrane extensions of the TGN loaded with E-cadherin from the Golgi and knock-down of golgin97 selectively blocked exit of E-cadherin cargo from the TGN.

There is a need to investigate golgin proteins as possible targets to modulate protein export and trafficking out of and within a cell.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

A list of Abbreviations is provided in Table 2.

The present invention identifies p230/golgin-245 (also referred to “p230”, “p230/golgin”, “golgin-245”) as an essential component in post-Golgi trafficking and exocytosis of TNFα from eukaryotic cells. Inhibition of TNFα trafficking reduces levels of precursor membrane bound TNFα and hence exogenous TNFα. Medicaments which target p230 or a molecule associated therewith are therefore useful in the treatment and prophylaxis of inflammatory diseases conditions in a subject.

Accordingly, one aspect of the present invention is a method for controling post-Golgi exocytosis of TNFα, the method comprising introducing to a cell an amount of an agent which modulates the function, activity, level or operability of p230/golgin-245 or a molecule associated therewith, the amount effective to inhibit or promote the ability of p230/golgin-245 to facilitate exocytosis of TNFα.

Whilst inhibition of TNFα exocytosis is desired for therapeutic purposes to reduce an inflammatory response, promotion of TNFα exocytosis is contemplated in animal disease model systems. Such systems are useful inter alia for screening potential anti-inflammatory drugs.

In a particular embodiment, the agent is an antagonist of p230/golgin-245. Hence, a method is provided for reducing post-Golgi exocytosis of TNFα from a cell, the method comprising contacting the cell with an antagonist of p230/golgin-245 in an amount effective to reduce p230/golgin-245-mediated IFNα exocytosis.

An “antagonist of p230/golgin-245” includes an antagonist of p230/golgin-245 function, level and/or activity and/or of a component associated therewith.

In another embodiment, a method is provided for enhancing post-Golgi exocytosis of TNFα from a cell, the method comprises contacting the cell with an agonist of p230/golgin-245 in an amount effective to enhance p230/golgin-245-mediated IFNα exocytosis. Such a method is useful in animal model systems to screen for anti-inflammatory drugs or to study the inflammatory response. An “agonist of p230/golgin-245” includes an agonist of p230/golgin-245 function, level and/or activity and/or of a component associated therewith.

Hence, the antagonists and agonists of the present invention may target p230 directly, expression of a gene encoding p230, multimer formulation and/or a molecule associated with p230 such as a G protein required for binding of p230 to a tubule. The antagonist (or agonist) may be administered to the cell or produced in the cell such as via a viral vector or via stem cells. The antagonists and agonists encompass small molecules, proteins and peptides and nucleic acid molecules.

The present invention is particularly directed to a method for the treatment or prophylaxis of inflammation in a subject, the method comprising administering to the subject an effective amount of an antagonist of p230/golgin-245-mediated TNFα exocytosis from cells of the subject.

A further aspect provides for the use of an antagonist of p230/golgin-245 in the manufacture of a medicament in the treatment of an inflammatory condition in a subject. In addition, the present invention contemplates the use of p230/golgin-245 in the manufacture of a medicament in the treatment of an inflammatory condition in a subject.

Particular subjects are primates such as humans.

Disease animal model systems are contemplated herein for testing of potential anti-inflammatory medicaments. Such systems may have reduced levels of p230 or p230 function or may over express p230. For example, the present invention provides an animal model comprising an elevated level of p230/golgin-245 and which is prone to inflammatory conditions. In one embodiment, the animal model is in the form of a retrogenic murine animal (e.g. mouse or rat) which expresses miRNA to silence the p230 gene. For example, stem cells may be genetically modified to express miRNA directed to the p230 gene and used to generate retrogenic animals.

TABLE 1
Sequence Identifiers

| Sequence Identifier | Sequence |
|---|---|
| 1 | Primer miRmp230a for RNAi designer |
| 2 | Primer miRmp230b for RNAi designer |
| 3 | Primer miRmGCC185 for RNAi designer |

TABLE 2
Abbreviations

| Abbreviation | Definition |
|---|---|
| Arl1 | G protein which recruits p230/golgin-245 and |
| | golgin-97 to TGN |
| GCC185 | Human TGN golgin |
| GCC88 | Human TGN golgin |
| Golgin | Long coiled protein from TGN |
| golgin-97 | Human TGN golgin |
| golgin-245 | p230/golgin-245 |
| LPS | Lipopolysaccharide |
| p230/golgin-245 | Human TGN golgin |
| p230 | p230/golgin-245 |
| RE | Recycling endosome |
| TGN | Trans-Golgi network |
| TNFα | Tumor Necrosis Factor Alpha |
| 245 | p230/golgin-245 |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIGS. 1a through d are photographic representations showing the TNFα trafficking is inhibited by silencing p230/golgin-245 in HeLa cells (a-c) HeLa cells transfected with control siRNA or p230 siRNA for 48 hrs and then transfected a second time with (a, b) YFP-TNFa and (c) GFP-Ecad for a further 24 h. In (b) myc-p230 was co-transfected with siRNA. Monolayers were then incubated with TACE inhibitor for 2 h, fixed in paraformaldehyde and cell surface TNFα stained with rabbit anti-TNFα antibodies followed by Alexa647-conjugated anti-rabbit IgG. Monlayers were then permeabilized and stained with (a, c) human anti-p230 antibodies followed by goat anti-human IgG or (b) monoclonal anti-myc antibodies followed by Alexa568-conjugated anti-mouse IgG. (d) HeLa were transfected with control siRNA or p230 siRNA for 48 hrs and lysed in SDS-PAGE reducing buffer and extracts subjected to SDS-PAGE on a 7.5% (w/v) polyacrylamide gel. Proteins were transfer to a PVDF membrane and probed with affinity purified rabbit anti-p230 antibodies using a chemiluminescence detection system. The membrane were then stripped and reprobed with anti-α-tubulin, followed by anti-golgin-97 antibodies. Bar represents 10 μm

FIGS. 2a and b are photographic and graphical representations showing TNFα in p230 labeled tubules leaving the TGN in live macrophages.

FIGS. 3a through c are photographic and graphic representations showing the Post-Golgi TNFa trafficking is inhibited by silencing p230 in stimulated RAW macrophages. RAW macrophages were transfected with control miRNA or p230 miRNA-1, as indicated, for 48 hrs and treated with 100 ng/ml of LPS in serum free RPMI at 37° C. for 2 hrs in the presence of presence of 10 μM of TAPI-1. Stimulated macrophages were then fixed in 4% (v/v) paraformaldehyde. (a) Fixed macrophages were permeabilized and stained with human anti-p230 antibodies followed by goat anti-human IgG and with rabbit anti-TNFa antibodies followed by Alexa647-conjugated anti-rabbit IgG. (b, c) Fixed macrophages were analyzed for cell surface TNFa by staining with rabbit anti-TNFα antibodies followed by Alexa647-conjugated anti-rabbit IgG. Representative flow cytometry plots of GFP+ cells shown in c. Bar represents 10 μm.

FIGS. 4a through d are photographic representations showing that Peritoneal macrophages from transgenic mice expressing p230 miRNA are depleted in p230 and impaired in TNFα secretion. Peritoneal macrophages obtained from either empty miRNA vector (control) or p230 miRNA retrogenic mice were fixed in 4% (v/v) paraformaldehyde and stained with (a) antihuman p230 antibodies followed by goat Alexa 594 conjugated anti-human IgG or (b) rabbit anti-human GCC88 antibodies or rabbit anti-human GCC185 antibodies, followed by goat Alexa 568 conjugated anti-rabbit IgG. (c,d) Peritoneal macrophages obtained from control and p230 miRNA transgenic mice were activated with 100 ng/ml of LPS in the presence of 50 nM of TAPI-1 for 2 hrs prior to fixation. Macrophage were fixed in 4% (v/v) paraformaldehyde and cell surface TNFα was detected using a rabbit anti-mouse TNFα antibodies, followed by goat Alexa 568 conjugated anti-rabbit IgG in non-permeabilized cells. (d) For internal TNFα detection, peritoneal macrophages were activated in 100 ng/ml of LPS for 2 hrs, fixed in 4% (v/v) paraformaldehyde and permeabilized, and stained with rabbit anti mouse TNFα antibodies, followed by goat anti rabbit Alexa 568 conjugated antibodies. p230/golgin-245 molecules were stained with affinity purified anti-human p230/golgin-245 antibodies, followed by goat anti-human Alexa 647 conjugated antibodies. Bar=10 μm.

### DETAILED DESCRIPTION

The present invention is predicated in part on the identification of a carrier of post-Golgi trafficking and exocytosis of IFNα. In particular, TGN golgin, p230/golgin-245, is required for transport of the membrane precursor of TNFα. The p230/golgin-245 carrier may also be referred to herein as “p230”, “245”, “golgin-245” or “p230/golgin”. Reference to this TGN golgin includes any and all of its homologs, orthologs, polymorphic variants, splice variants and natural and artificially induced derivatives. It also includes multimeric foul's such as homo- and hetero-dimers comprising a p230 monomer.

p230/golgin-245 and co-factors or associated molecules all form targets to inhibit p230-mediated post-Golgi TNFα exocytosis. The ability to control TNFα secretion by selective silencing of trafficking machinery has a range of applications including controling inflammatory processes.

Hence, one aspect of the present invention contemplates a method for controling post-Golgi exocytosis of TNFα, the method comprising introducing to a cell an amount of an agent which modulates the function, activity, level or operability of p230/golgin-245 or a molecule associated therewith the amount effective to inhibit or promote the ability of p230/golgin-245 to facilitate exocytosis of TNFα.

Reference to “TNFα” includes its homologs, orthologs, polymorphic variants and derivatives.

The “cell” is generally a eukaryotic cell and in particular a mammalian cell such as but not limited to a macrophage, monocytes, dendritic cell, lymphocyte or other cells of the immune system or their precursors. Generally, the mammal is a human or other primate. However, the present invention extends to veterinary and animal husbandry applications and hence the mammal may also be a livestock animal, companion animal or captive wild animal.

Examples of molecules associated with p230 include those molecules which are required for binding of the p230 to the tubule of the TGN. One example of a molecule is the G protein, Arl1. Hence, the agent may target inter alia p230, Arl1, p230 interaction with Arl1 and p230/Arl interaction with the membrane of the tubule or may target a gene encoding any of those components.

As indicated above, the ability to control p230 function enables inflammatory processes to be modulated and in a particular embodiment, inhibited.

Accordingly, another aspect provides a method for reducing post-Golgi exocytosis of TNFα from a cell, the method comprising contacting the cell with an antagonist of p230/golgin-245 in an amount effective to reduce p230/golgin-245-mediated IFNα exocytosis.

Reference to an “antagonist of p230/golgin-245” includes an antagonist of p230/golgin-245 function or level or activity. Inhibiting p230/golgin-245 function may also include inhibiting a component which associates with p230. An “agonist of p230/golgin-245” includes an agonist of p230/golgin-245 function or level or activity or of a component associated therewith.

Another aspect contemplates a method for ameliorating the effects of an inflammatory disease or condition in a subject, the method comprising administering to the subject an effective amount of an agent which reduces the function or level of p230/golgin-245 or a molecule associated therewith the administration being for a time and under conditions sufficient to reduce TNFα-mediated inflammatory processes.

Examples of inflammatory disease conditions contemplated by the present invention include but are not limited to those diseases and disorders which result in a response of redness, swelling, pain, and a feeling of heat in certain areas that is meant to protect tissues affected by injury or disease. Inflammatory diseases which can be treated using the methods of the present invention, include, without being limited to, acne, angina, arthritis, asthma, aspiration pneumonia disease, chronic obstructive pulmonary disease (COPD), colitis, empyema, gastroenteritis, intestinal flu, necrotizing enterocolitis, pelvic inflammatory disease, pharyngitis, pleurisy, raw throat, rubor, sore throat, urinary tract infections, chronic inflammatory demyelinating polyneuropathy, chronic inflammatory demyelinating polyradiculoneuropathy. Pathogenic infection such as by Leischmonia may also be treated. COPD, asthma and colitis are particularly useful targets for the medicaments contemplated herein.

The terms “inflammation”, “inflammatory response”, inflammatory condition” and “inflammatory disease” are used interchangeably throughout this specification. Generally, the inflammatory response is regarded as being caused by, associated with or exacerbated by, TNFα.

The present invention provides, therefore, agents which modulate either the level of p230 gene or the activity of a gene encoding p230 or the activity or level of a molecule associated with p230 (such as a G protein required for coupling of p230 to a tubule membrane) for use in the treatment and prophylaxis of inflammation or inflammatory conditions such as asthma, COPD or colitis. The agents are conveniently in a composition comprising the agent and one or more pharmaceutically acceptable carriers, diluents and/or excipients. Two or more agents may be co-administered in the same composition or in separate compositions.

Notwithstanding, agents which are agonists of p230 functions are also contemplated to assist in animal disease model systems.

The agents include antagonists and agonists and may be administered to the cell or produced in the cell via for example, viral vectors. Examples of antagonists include intracellular antibodies, RNA species (e.g. miRNA siRNA, dsRNA, ssRNA) and small molecules which cross cellular membranes.

A method is provided for enhancing post-Golgi exocytosis of TNFα from a cell, the method comprises contacting the cell with an agonist of p230/golgin-245 in an amount effective to enhance p230/golgin-245-mediated IFNα exocytosis. In addition, the present invention provides an animal model comprising an elevated level of p230/golgin-245 and which is prone to inflammatory conditions.

Unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

As used in the subject specification, the singular forms “a”, “an” and “the” include plural aspects unless the context clearly dictates otherwise. Thus, for example, reference to “a cell” includes reference to a single cell or more than one cell; reference to “an active agent” includes a single active agent, as well as two or more active agents; reference to “the invention” includes reference to single or multiple aspects of an invention; and so forth.

The terms “compound”, “active agent”, “pharmacologically active agent”, “medicament”, “active” and “drug” are used interchangeably herein to refer to a chemical compound that induces a desired biological effect. This biological effect includes modulating the level or activity or function of p230/golgin-245 or any molecule associated therewith such as a G-protein (e.g. Arl1) or co-factor or a monomer involved in a multimeric (e.g. dimer) complex comprising p230. Although generally the dimmers or other multimers are generally homo-multimers, hetero-multimers are also contemplated herein. The biological effect may also be a reduced level of exogenous TNFα or membrane-associated precursor TNFα. The effect may also be an amelioration of symptoms of inflammation. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

The term “compound” is not to be construed as a chemical compound only but extends to peptides, polypeptides and proteins as well as genetic molecules such as RNA, DNA and chemical analogs thereof. An RNA species, for example, includes miRNA, SiRNA, dsRNA and ssRNA. The term “modulator” is an example of a compound, active agent, pharmacologically active agent, medicament, active and drug which up-regulates or down-regulated either the level of expression of the p230 gene or the activity of p230 (or a molecule associated therewith). The term “down-regulates” encompasses the inhibition, reduction or prevention of expression of the p230 gene or of the activity of p230, so as to correspondingly reduce an inflammatory response or the risk of an inflammatory response being elicited. Such a modulator may be referred to herein as an “inhibitor” or antagonist. Similarly, the term “up-regulates” encompasses the induction, increase or potentiation of expression of p230 gene or of the activity of p230, so as to correspondingly enhance an inflammatory response or the risk of an inflammatory response being elicited. Such a modulator may, therefore, be referred to herein as a “potentiator” or agonist. The latter class of agents are likely to be useful in model disease systems to test for anti-inflammatory agents.

The present invention contemplates, therefore, compounds useful in modulating either the level of expression of a p230 gene or the activity of the p230 or of a molecule associated therewith. The compounds, when antagonists, have an effect on reducing or preventing or treating inflammatory conditions. Reference to a “compound”, “active agent”, “pharmacologically active agent”, “medicament”, “active” and “drug” includes combinations of two or more actives such as one or more inhibitors and/or potentiators. A “combination” also includes a two-part or more such as a multi-part pharmaceutical composition where the agents are provided separately and given or dispensed separately or admixed together prior to dispensation.

The terms “effective amount” and “therapeutically effective amount” of an agent as used herein mean a sufficient amount of the agent to provide the desired therapeutic or physiological effect. Ultimately, as far as an inhibitor/antagonist is concerned, the desired physiological effect is a reduction in TFNα-mediated inflammation. The agent may induce or prevent the expression of a p230 gene; act as an antagonist of p230; act as an antagonist of a co-factor of p230 or a molecule required by p230 to bind to a tubule, inter alia. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective, amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of an inflammatory disease or condition in a subject as well as treatment of a clinically symptomatic subject by inhibiting or causing regression of an inflammatory condition or disorder. Generally, such a condition or disorder is an inflammatory response or mediates or facilitates an inflammatory response or is a downstream product of an inflammatory response. Thus, for example, the present method of “treating” a patient with an inflammatory condition or with a propensity for one to develop encompasses both prevention of the condition, disease or disorder as well as treating the condition, disease or disorder. In any event, the present invention contemplates the treatment or prophylaxis of any inflammatory-type condition and, in particular, an inflammatory condition exacerbated by TNFα.

“Patient” or “subject” as used herein refers to an animal, particularly a mammal and more particularly human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, patient, animal, host or recipient. As indicated above, the compounds and methods of the present invention have applications in human medicine, veterinary medicine as well as in general, domestic or wild animal husbandry.

The compounds of the present invention may be large or small molecules, nucleic acid molecules (including antisense or sense molecules and microRNAs), peptides, polypeptides or proteins or hybrid molecules such as RNAi- or siRNA-complexes, ribozymes or DNAzymes. The compounds may need to be modified so as to facilitate entry into a cell.

The present invention provides, therefore, medicaments which modulate either the level of p230 gene expression or the activity of p230 or a molecule associated therewith which modulate levels or activities of inhibitors or potentiators of p230. Furthermore, the present invention contemplates the use of p230/golgin-245 in the manufacture of a medicament for the treatment of an inflammatory condition in a subject.

The present invention contemplates, therefore, methods of screening for medicaments comprising, for example, contacting a candidate drug with p230 or a gene encoding same. For convenience, the term “target” is used to collectively describe p230, its gene or a molecule (or gene) associated with p230 such as Arl1. The screening procedure includes assaying (i) for the presence of a complex between the drug and target, or (ii) for an alteration in the expression levels of a target gene.

One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the agent rather than the target and to measure the amount of agent binding the target in the presence and in the absence of the drug being tested. Such compounds may inhibit the target which is useful, for example, in finding inhibitors of p230.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase.

The present invention also contemplates the use of competitive drug screening assays in which neutralizing antibodies capable of specifically binding the target compete with a test compound for binding to the target or fragments thereof. In this manner, the antibodies can be used to detect the presence of any peptide which shares one or more antigenic determinants of the target.

Analogs of p230 may also be useful as antagonists. These analogs may compete for G-proteins required for binding to the tubule membrane.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Another useful group of compounds is a mimetic. The terms “peptide mimetic”, “target mimetic” or “mimetic” are intended to refer to a substance which has some chemical similarity to p230 but which antagonises or agonises or mimics p230. A peptide mimetic may be a peptide-containing molecule that mimics elements of protein secondary structure (Johnson et al, “Peptide Turn Mimetics” in Biotechnology and Pharmacy, Pezzuto et al, Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to p230. Peptide or non-peptide mimetics may be useful, for example, to inhibit p230 activity or to compete with any molecules associated therewith.

A substance identified as a modulator of p230 expression or p230 activity or function may be a peptide or non-peptide. Non-peptide “small molecules” are often preferred for many in vivo pharmaceutical uses. Accordingly, a mimetic or mimic of the peptide may be designed for pharmaceutical use.

The designing of mimetics of p230 to a pharmaceutically active compound is a known approach to the development of pharmaceuticals based on a “lead” compound. Mimetic design, synthesis and testing are generally used to avoid randomly screening large numbers of molecules for a desired property.

There are several steps commonly taken in the design of a mimetic of p230. First, the particular parts of p230 that are critical and/or important in conferring function are determined. This can be done by systematically varying the amino acid residues in the protein, e.g. by substituting each residue in turn. Alanine scans of polypeptide are commonly used to refine such motifs. These parts or residues constituting the active region of p230 are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the pharmacophore and/or its binding partner are modeled. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. In addition, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of p230 or of small molecules with which they interact (e.g. agonists, antagonists, inhibitors or enhancers) in order to fashion drugs which are, for example, more active or stable forms of the polypeptide, or which, e.g. enhance or interfere with the function of p230 in vivo. See, e.g. Hodgson (Bio/Technology 9:19-21, 1991).

It is also possible to use a p230-specific antibody, and then to generate an anti-idiotypic antibody (anti-ids) As a mirror image of the p230 binding site of the first mentioned antibody, the binding site of the anti-ids would be expected to be an analog of the binding site. The anti-id could then be used to identify and isolate peptides from banks of chemically or biologically produced banks of peptides. Selected peptides would then act as the pharmacore.

Two-hybrid screening is also useful in identifying co-factors of p230. Two-hybrid screening conveniently uses, for example, Saccharomyces cerevisiae and Saccharomyces pombe. This approach screens for ligands of p230 and takes advantage of transcriptional factors that are composed of two physically separable, functional domains. The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (e.g. lacZ) produces a detectable phenotype. In the present case, for example, S. cerevisiae is co-transformed with a library or vector expressing a cDNA GAL4 activation domain fusion, and a vector expressing the p230 gene fused to GAL4. If lacZ is used as the reporter gene, co-expression of the fusion proteins will produce a blue color. Small molecules or other candidate compounds which interact with p230 will result in loss of color of the cells. Reference may be made to the yeast two-hybrid systems as disclosed by Munder et al, (Appl. Microbiol. Biotechnol. 52(3):311-320, 1999) and Young et al, Nat. Biotechnol. 16(10):946-950, 1998).

Another useful potential inhibitor of p230 is a cartilaginous fish-derived immunoglobulin-like molecule which binds to p230 or a co-factor thereof. More particularly, the immunoglobulin-like molecule comprises the variable domain of an IgNAR (Immunoglobulin new antigen receptor), referred to as “V_{NAR}”. The immunoglobulin-like molecules of the present invention enable the selective targeting of p230 and its precursor or processed forms which include monomeric or multimeric forms thereof or of molecules associated therewith.

Accordingly, the present invention provides an isolated, cartilaginous fish-derived immunoglobulin-like molecule which binds to p230/golgin-245.

In a particular embodiment, the immunoglobulin-like molecule comprises a variable domain of an IgNAR, referred to herein as V_{NAR}. IgNARs are described in International Patent Application No. WO 2005/118629.

IgNARs are classified in relation to their time of appearance during fish development and disulfide bonding patterns within variable domains. The categories are Type I V_{NAR}, Type 2 V_{NAR} and Type 3 V_{NAR} (Nuttal et al, Mol. Iminunol. 38:313-316, 2001; Nuttal et al, Eur j Biochem 270:3543-3554, 2003). Hence, the present invention encompasses an isolated Type 1 or 2 or 3 V_{NAR} from an IgNAR which binds to HBeAg and/or HBcAg or a precursor or processed form thereof.

Reference to a “cartilaginous fish” includes a member of the families of shark and ray. Reference to a “shark” includes a member of order Squatiniformes, Pristiophoriformes, Squaliformes, Carcharinformes, Laminiformes, Orectolobiformes, Heterodontiformes and Hexanchieformes. Whilst not intending to limit the shark to any one genus, immunoglobulins from genus Orectolobus are particularly useful and include the bamboo shark, zebra shark, blind shark, whale shark, nurse shark and Wobbegong. Immunoglobulins from Orectolobus maculates (Wobbegong) are exemplified herein.

The “immunoglobulins” from cartilaginous fish may be referred to herein as “immunoglobulin-like” to emphasize that the cartilaginous fish-derived molecules are structurally different to mammalian or avian-derived immunoglobulins. See Nuttal et al, 2003 supra. For brevity, all cartilaginous fish-derived immunoglobulin-like molecules are referred to herein as “IgNARs”. The variable domain from an IgNAR is referred to as a V_{NAR}.

Reference to “derived” includes vaccination of a fish and collection of blood or immune sera or other body fluid as well as the generation of molecules via recombinant means. By “recombinant means” includes generation of cartilaginous fish-derived nucleic acid libraries and biopanning expression libraries (such as phagemid libraries) for IgNAR proteins which interact with p230.

The present invention extends to a genetic approach to down-regulating expression of the p230 gene. In one example, nucleic acid molecules that induce temporary or permanent silencing of the p230 gene may be used to reduce levels of p230.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts of the p230 gene. Furthermore, polynucleotide vectors containing all or a portion the p230 gene may be placed under the control of a promoter in an antisense orientation and introduced into a cell. Expression of such an antisense construct within a cell will interfere with target transcription and/or translation. Furthermore, co-suppression and mechanisms to induce RNAi or siRNA or microRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7:187-195, 1997). Such compounds can also be injected into embryos and the effect of interference with mRNA observed.

In one embodiment, the present invention employs compounds such as oligonucleotides and similar species for use in modulating the function or effect of the p230 gene, i.e. the oligonucleotides induce pre-transcriptional or post-transcriptional gene silencing. This is accomplished by providing oligonucleotides which specifically hybridize with a p230 gene transcript. The oligonucleotides may be provided directly to a cell or generated within the cell. As used herein, the term “target nucleic acid” is used for convenience to encompass DNA encoding the p230 transcript (including pre-mRNA and mRNA or portions thereof).

In an alternative embodiment, genetic constructs including DNA vaccines are used to generate sense or antisense molecules in vivo.

Following identification of an agent which modulates the level of expression of the p230 gene or p230, it may be manufactured and/or used in a preparation, i.e. in the manufacture or formulation or a composition such as a medicament, pharmaceutical composition or drug. These may be administered to individuals in a method of treatment or prophylaxis. Alternatively, they may be incorporated into a patch or slow release capsule or implant or incorporated into a microparticle, inhalant spray or otherwise suitable medium.

Thus, the present invention extends, therefore, to a pharmaceutical composition, medicament, drug or other composition including a patch or slow release formulation or inhalant formulation comprising an agonist or antagonist of p230 gene or p230. Another aspect of the present invention contemplates a method comprising administration of such a composition to a patient such as for treatment or prophylaxis of an inflammatory condition. Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days. Preferably, within seconds or minutes.

Two- or multi-part pharmaceutical compositions or packs are also contemplated with multiple components, such as comprising those which down-regulate or up-regulate the level of expression of the p230 gene or the activity of p230.

Accordingly, another aspect of the present invention contemplates a method for the treatment or prophylaxis of an inflammatory condition in an animal, the method comprising administering to the animal an effective amount of a compound as described herein or a composition comprising same.

The term “administering to” includes the inhalant or nasal application of a composition.

This method also includes providing a wild-type or mutant target gene function to a cell. This is particularly useful when generating an animal model. Alternatively, it may be part of a gene therapy approach. This may be particularly useful when an infant or fetus comes from one or more parents which are likely to pass on the genetic predisposition of, for example, asthma. A target gene or a part of the gene may be introduced into the cell in a vector such that the gene remains extrachromosomal. In such a situation, the gene will be expressed by the cell from the extrachromosomal location. If a gene portion is introduced and expressed in a cell carrying a mutant target allele, the gene portion should encode a part of the target protein. Vectors for introduction of genes both for recombination and for extrachromosomal maintenance are known in the art and any suitable vector may be used. Methods for introducing DNA into cells such as electroporation calcium phosphate co-precipitation and viral transduction are known in the art.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al, J. Gen. Virol. 73:1533-1536, 1992), adenovirus (Berkner, Curr. Top. Microbiol. Immunol. 158:39-66, 1992; Berkner et al, BioTechniques 6:616-629, 1988; Gorziglia and Kapikian, J. Virol. 66:4407-4412, 1992; Quantin et al, Proc. Natl. Acad. Sci. USA 89:2581-2584, 1992; Rosenfeld et al, Cell 68:143-155, 1992; Wilkinson et al, Nucleic Acids Res. 20:2233-2239, 1992; Stratford-Perricaudet et al, Hum. Gene Ther. 1:241-256, 1990; Schneider et al, Nature Genetics 18:180-183, 1998), vaccinia virus (Moss, Curr. Top. Microbiol. Immunol. 158:25-38, 1992; Moss, Proc. Natl. Acad. Sci. USA 93:11341-11348, 1996), adeno-associated virus (Muzyczka, Curr. Top. Microbiol. Immunol. 158:97-129, 1992; Ohi et al, Gene 89:279-282, 1990; Russell and Hirata, Nature Genetics 18:323-328, 1998), herpesviruses including HSV and EBV (Margolskee, Curr. Top., Microbiol. Immunol. 158:67-95, 1992; Johnson et al, J. Virol. 66:2952-2965, 1992; Fink et al, Hum. Gene Ther. 3:11-19, 1992; Breakefield and Geller, Mol. Neurobiol. 1:339-371, 1987; Freese et al, Biochem. Pharmacol. 40:2189-2199, 1990; Fink et al, Ann. Rev. Neurosci. 19:265-287, 1996), lentiviruses (Naldini et al, Science 272:263-267, 1996), Sindbis and Semliki Forest virus (Berglund et al, Biotechnology 11:916-920, 1993) and retroviruses of avian (Bandyopadhyay and Temin, Mol. Cell. Biol. 4:749-754, 1984; Petropoulos et al, J. Viol. 66:3391-3397, 1992], murine [Miller, Curr. Top. Microbiol. Immunol. 158:1-24, 1992; Miller et al, Mol. Cell. Biol. 5:431-437, 1985; Sorge et al, Mol. Cell. Biol. 4:1730-1737, 1984; and Baltimore, J. Virol. 54:401-407, 1985; Miller et al, J. Virol. 62:4337-4345, 1988] and human [Shimada et al, J. Clin. Invest. 88:1043-1047, 1991; Helseth et al, J. Virol. 64:2416-2420, 1990; Page et al, J. Virol. 64:5270-5276, 1990; Buchschacher and Panganiban, J. Virol. 66:2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viralvectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

Cells and animals which carry mutant p230 alleles or where one or both alleles are deleted can be used as model systems to study the effects of modulating the expression of the p230 gene, and/or the activity of p230, on inflammation. Mice, rats, rabbits, guinea pigs, hamsters, zebrafish and amphibians are particularly useful as model systems. A particularly useful insertion is a loxP sequence flanking a target gene which can be excised by cre. Alternatively, the model system may be a tissue culture system. An “animal model” may, therefore, be tissues from an animal.

The present invention provides, therefore, a mutation in or flanking a genetic locus encoding p230. The mutation may be an insertion, deletion, substitution or addition to the p230-coding sequence or its 5′ or 3′ untranslated region.

The animal model of the present invention is useful for screening for agents capable of ameliorating or mimicking the effects of p230. In one embodiment, the animal model produces low amounts of a p230. In another animal model, excess p230 is produced.

The compounds, agents, medicaments, nucleic acid molecules and other target antagonists or agonists of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698. Microparticle sprays, inhalants and fumes are particularly useful compositions.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention is further described by the following non-limiting Examples. Materials and Methods used in these Examples are provided below.

### Antibodies, Plasmids and Reagents

Plasmids construct containing YFP-TNFα and GFP-Ecadherin were previously described (Lock et al, Traffic 6(12):1142-1156, 2005). Myc tagged full length p230 have been previously described (Erlich et al, J Biol Chem 271 (14):8328-8337, 1996). To generate retroviral DNA constructs expressing microRNA, PCR amplification was used to amplify the microRNA insert from pcDNATM 6.2 GW/EmGFP expression vector and then subcloned into pMIG-MSCV vector to produce pMIG-MSCV mp 230a or mp 230b constructs.

The following primary antibodies were used:Human autoantibodies to p230 and affinity purified rabbit polyclonal antibodies to p230/golgin245 have been previously described. Rabbit polyclonal antibodies to human GCC88 and GCC185 were prepared by standard procedures. Mouse monoclonal antibody to GM130 and golgin-97 were purchased from BD Biosciences (NSW, Australia). Mouse monoclonal anti α-tubulin was obtained from Amersham, UK. A rabbit polyclonal antibody to mouse TNFα was purchased from Chemicon (Millipore, NSW, Australia). The 9E10 mouse monoclonal antibody specific for the myc epitope has been described. HECD1 a mouse monoclonal antibody was used to detect human E cadherin. Murine MHC class II were detected using anti-I-E antibodies (clone 14-4-4S) from Escherichia coli 011:B4 was purchased from Sigma Adrich (NSW, Australia). TACE inhibitor TAPI-1 was purchased from Calbiochem (Merck, Victoria, Australia). Secondary antibodies used for immunofluorescence were goat anti-rabbit IgG-Alexa Fluor (Trade Mark) 568, goat anti-rabbit IgG-Alexa Fluor (Trade Mark) 488, Goat anti-human Alexa Fluoro (Trade Mark) 647 nm and goat anti-human Alexa Fluor (Trade Mark) 594 nm were from Molecular Probes (Invitrogen, Carlsbad, Calif., USA). Horse-radish peroxidase-conjugated sheep anti-rabbit Ig and anti-mouse Ig were from DAKO Corporation (Carpinteria, Calif., USA)

### Cell Culture and Transfection

HeLa cells and 3T3 mouse fibroblasts were maintained as semi-confluent monolayer in Dulbecco's Modified Eagle's media (DMEM) supplemented with 10% (v/v) fetal calf serum (FCS), 2 mM L-glutamine, 100 units/μl. RAW264.7 murine macrophages were cultured in RPMI 1640 medium (Invitrogen, Carlsbad, Calif., USA) containing 10% (v/v) heat inactivated serum supreme (BioWhittaker, Australia) and 1% (w/v) Lglutamine. For transfections, HeLa cells and 3T3 mouse fibroblast were seeded as monolayers and transfected using Fugene 6 (Roche Diagnostic, Basel, Switzerland) according to manufacturer's instructions. Transfections were carried out in C-DMEM at 37° C., 10% (v/v) CO₂ for 24-96 hrs. Transient transfection of siRNA was performed using Oligofectamine (Invitrogen, Carlsbad, Calif., USA) according to manufacturer's instruction for 72 hrs at 37° C. prior to analysis. RAW 264.7 murine macrophages were transfected with miRNA constructs either by electroporation or using Lipofactamine 2000 (Invitrogen). For electroporation, 2.5×10⁷ cells were mixed with 20 μg of DNA with a high capacitance setting (240V and 950 μF on exponential decay setting) using an electroporation system (Gene Pulser II; BioRad Laboratories). Cells were then washed in cold SF RPMI, plated out on non-coated 10 cm plates in warmed C—RPMI with 10% (v/v) serum supreme and incubated at 37° C. for 96 hrs. For Lipofectmine 2000 transfection, 2 μg of DNA was mixed with 10 μl of Lipofectamine 2000 and then added to 2.5×10⁷ cells in the presence of Optimum (Invitrogen, Carlsbad, Calif., USA) at 37° C. overnight. Optimun medium in transfected macrophages were replace with C—RPMI the next day and incubate at 37° C. for another 72 hrs.

siRNA and miRNA

Mouse and human p230/golgin245 and human GCC88 was targeted with siRNA duplex.

For knockdown of mouse p230/golgin245 using a miRNA system (Invitrogen) the following primer sets were designed using Invitrogen BLOCK-iT (Trade Mark) RNAi designer, annealed and cloned into pcDNA (Trade Mark) 6.2 GW/EmGFP miR expression vector containing a GFP expression cassette according to manufacturer's instructions.

miRmp230a
(SEQ ID NO: 1)
5′TGCTGAATAGCGTCGGCTTTGTCACGGTTTTGGCCACTGACTGAC
CGTGACAACCGACGCTATT-3′
miR mp230b
(SEQ ID NO: 2)
5′TGCTGAATTGTTACACTGTCCTTGGTGTTTTGGCCACTGACTGAC
ACCAAGGAGTGTAACAATT-3′
miR mGCC185
5′TGCTGTAAGATGGCCGTTTCTTTGCTGTTTTGGCCACTGACTGAC
AGCAAAGACGGCCATCTTA-3′

### Assay for TNFα Secretion by Activated Macrophage

The trafficking of TNFα from the Golgi to the cell surface was measured. Briefly, macrophages were activated with 100 ng/ml of LPS in serum free RPMI at 37° C. for 2-4 hrs and fixed in 4% (v/v) PFA to stop activation. For internal staining of TNFα, macrophages were fixed in 4% (v/v) PFA, permeabilized in 0.1% (v/v) triton x-100 and then stained for mouse TNFα. To detect for cell surface TNFα, activated macrophages were stimulated as described above in the presence of 10 μM of TAPI-1, fixed in 4% (v/v) PFA, stained with rabbit polyclonal antibodies to mouse TNFα in non-permeabilized cells. For FACS quantification of cell surface TNFα, macrophages were activated in the presence of 10 μM of TAPI-1 as described above, stained with rabbit polyclonal antibody to mouse TNFα and then FACS analyzed by gating on GFP+ cells.

### Generation of Retroviral Producer Cells and Stable Transduction of 3T3

293T cells were transiently cotransfected with the murine stem cell vector containing microRNA and its packaging plasmids. Retroviral producer cell lines were then generated by repeatedly transducing GP+E86 cells (6-8 times) with viral supernatant harvested from 293T transfection. To test viral titre of retroviral producer lines, 3T3 fibroblast were transduced with supernatant harvested from producer cells in the presence of polybrene (hexadimethrine bromide; 6 μg/ml) for 72 hrs. Transduced cells were analyzed for GFP expression by FASC analysis.

Generation of p230 Depleted Retrogenic Mice

Bone marrow was harvested from 6-8 weeks old donor mice, 48 hrs after treatment with 150 mg/kg of 5-fluoruracil (Sigma). Bone marrow cells were cultured in C-DMEM with 20% (v/v) FCS and the stem cells induced to proliferate with 20 ng/ml murine interleukin-3 (mIL-3), 50 ng/ml of human interleukin-6 (hIL-6) and 50 ng/ml of murine stem cell factor (mSCF) (Invitrogen, Biosource international). Bone marrow cells were co-culture for 48 hrs with the retroviral produced lines described above. The nonadherent, transduced bone marrow cells were collected and washed. Sub lethally irradiated (600rad-750rad) recipient mice were injected via the tail vein with 4×106 bone marrow cells with 2% (v/v) FCS and 20 u/ml of heparin (Sigma). Mice were analyzed 8-10 week post transplant.

### Isolation of Peritoneal Macrophage

8 to 10 weeks old reconstituted retrogenic mice or wild type Balb/C mice were injected with 8 ml of prewarmed C—RPMI into their peritoneal cavity. Macrophages were liberated by massage and the medium recollected back into the syringe. Peritoneal macrophages were washed once in warm C—RPMI, plated on coverslips and incubate overnight at 37° C. After incubation, non-adherent cells were then washed off with CRPMI.

### Indirect Immunofluorescence

Cells were fixed in 4% (v/v) paraformaldehyde for 15 min, followed by quenching in 50 mM NH4Cl/PBS for 10 min. Cells were either permeabilized by 0.1% (v/v) Triton X-100/PBS or 0.1% (v/v) saponin/PBS for 4 mins. Cell monolayer were blocked in PBS containing 5% (v/v) fetal calf serum for 20 mins to reduce non-specific binding. Monolayers were incubated in primary antibodies, diluted in 5% FCS/PBS for 1 h at room temperature. Cells were hen washed 6 times in PBS over 30 mins, before fluorochrome conjugate, antibodies were added and incubated for 30 mins at room temperature. Washes were carried out as above. Monolayers were then rinsed with milliQ water before mounting in Mowiol. Confocal microscopy was performed using a Leica TCS SP@ imaging system. For multi-color labeling, images were collected separately. To quantitate cell surface TNFα in activated peritoneal macrophages, images were collected on the same confocal settings for both control (empty) vector (n=46) and p230 miRNA macrophages (n=46) at each timepoint after LPS stimulation. Total fluorescence intensity for GFP and TNFα were determined by Leica confocal software (version beta 2000). GFP-intensity values were used to categorize macrophages into weak GFP positive cells, medium GFP positive cells and bright GFP positive cells before analysis of TNFα fluorescence intensity. Results were expressed as means and p-value were determined by student t-test.

### Immunoblot

Cell extracts were obtained by resuspending cell pellet in 4× reducing sample buffer. Protein samples were resolved on a 4-12% (w/v) NuPAGE gradient gel (Invitrogen) according to manufacturer's instructions and transferred overnight onto a polyvinylidene fluoride membrane (Millipore, NSW Australia). The membrane was blocked by drying at room temperature. The blocked membrane was incubated with primary antibodies, diluted in 1% (w/v) Blotto (1% (w/v) Skim milk powder in PBS) for 1 h with rocking before the addition of HRP-conjugated secondary antibodies for 30 min. The membrane was then rinsed with 0.05% (w/v) Tween-20/PBS before and after incubation with primary and secondary antibodies. Bound antibodies were detected by chemiluminescence (NEN, Boston, USA) and captured using the Gel Pro analyzer program (MediaCybernetics, Bethesda, Md. USA). Densitometry of the protein bands were measured using the Gel Pro analyzer program (MediaCybernetics, Bethesda, Md.).

### Example 1

### TGN Golgin, p230 is Required for TNFα Secretion in HeLa Cells

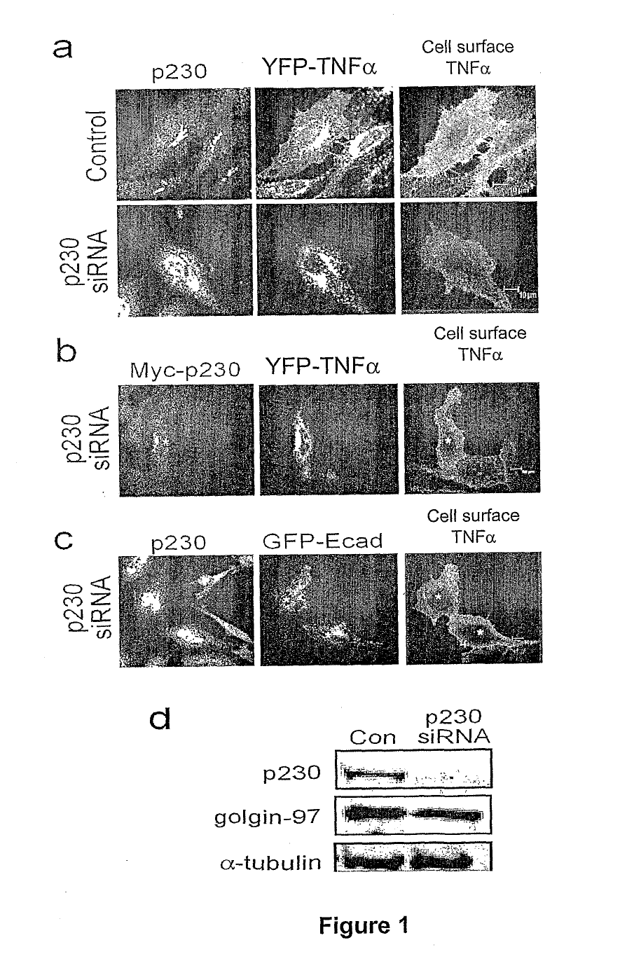

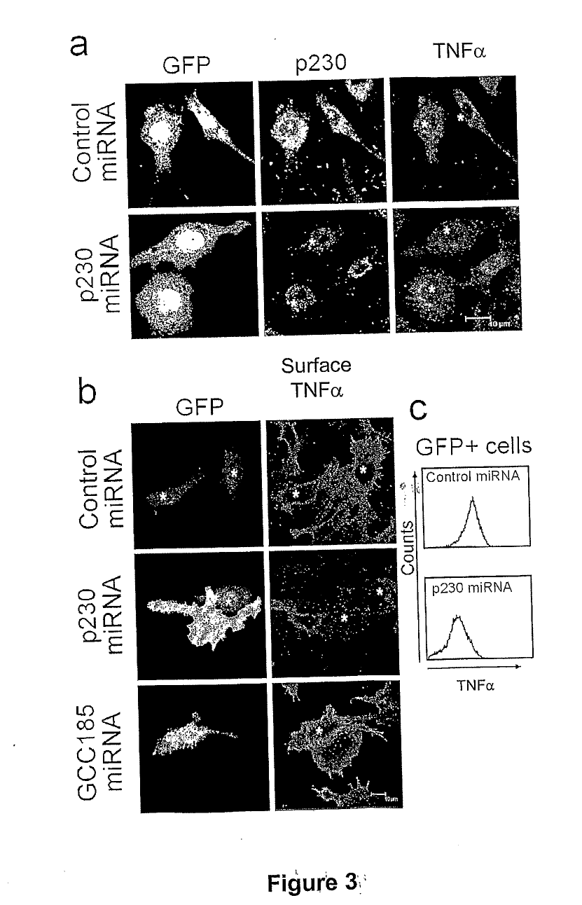

Golgins mark different subdomains of the TGN and tubules arising from these subdomains have different golgins associated with them. To determine if p230 was required for post-Golgi export of TNFα, HeLa cells were depleted of p230 using siRNA and then transfected with TGN38-YFP. p230 was depleted to >75% in siRNA transfected cells, whereas the related TGN golgin, golgin-97, was unaffected (FIG. 1d). In both control and p230-depleted cells, YFP-TNFα showed intracellular perinuclear Golgi-like staining (FIG. 3). To allow detection of TFNα at the cell surface, a TNFα converting enzyme (TACE) inhibitor was included to block proteolytic release of surface TNFα. Cell surface TNFa was readily detected on non-permeabilized control cells (FIG. 1), however, there was very little surface TNFα detected on p230 siRNA transfected cells. In contrast, and as expected from our previous findings, the membrane cargo E-cadherin was efficiently transport to the plasma membrane of p230-depleted cells (FIG. 1c). Depletion of another TGN golgin, GCC88, has no affect on cell surface transport of TNFα, demonstrating that the block in TNFα export was p230 specific.

To rule out the possibility of off-target affects by the siRNA, p230-depleted HeLa were transfected with a myc-tagged full-length p230 construct (myc-p230) to determine if overproduction of wild-type p230 would rescue the observed block in TNFa transport. In p230 depleted HeLa expressing myc-p230FL, cell surface TNFα was readily detected in all cells examined (>20 cells analyzed) (FIG. 1b), indicating that full length exogenous p230 protein rescued the block in TNFα export from the Golgi.

### Example 2

### Characterization of TNFα in p230 Tubules Leaving the TGN in Live Macrophages

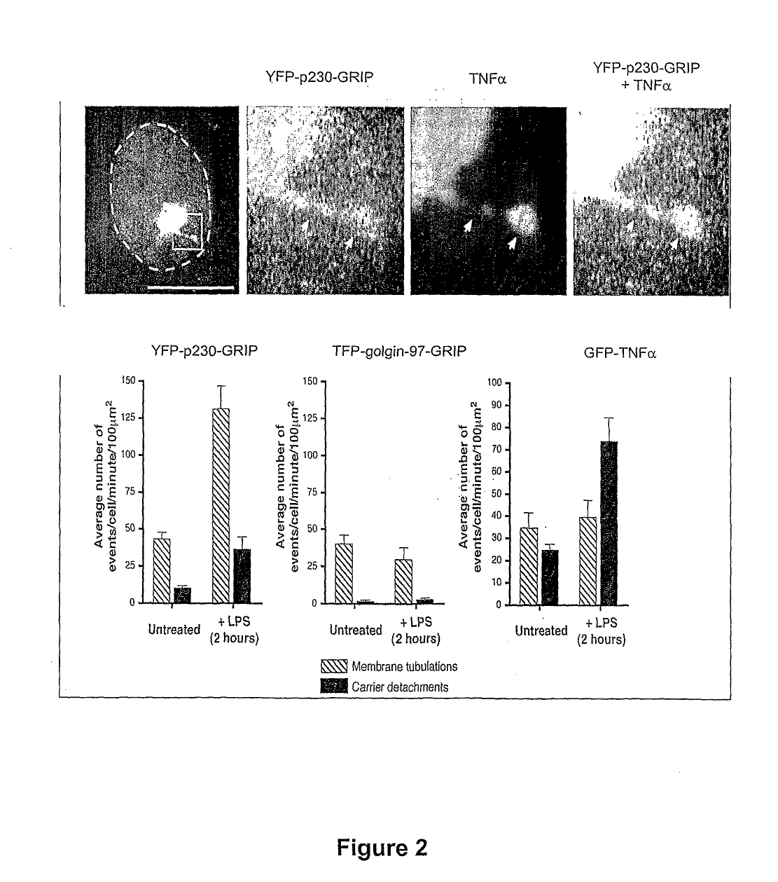

Newly-synthesized cytokines, including the transmembrane precursor of TNFα, initially accumulate in the Golgi complex and are then loaded into carriers which bud off the TGN for post-Golgi transport and secretion. Having established a role for p230 in the post-Golgi export in HeLa cells, the relevance of these findings in macrophages was examined. LPS-stimulated RAW264.7 macrophages cells were examined for the relationship between p230 and TFNα at the TGN. Macrophages were transiently transfected with GFP-TNFα and/or with YFP-labeled GRIP domains of p230 or golgin97 TGN-derived tubules and budding carriers were viewed in live macrophages and also analyzed by immunolabeling in a series of fixed cells. Typically, endogenous TNFα or GFP-TNFα was seen emerging from the TGN as a bolus in tubules labeled with the YFP-p230GRIP (FIG. 2a). The transport of TNFα from the TGN to the recycling endosome involves the SNARE complex of syntaxin6/syntaxin7/Vtilb and accordingly p230 tubules can be seen colabeled with syntaxin6. Neither endogenous TNFα nor GFP-TNFα in macrophages was seen associated with golgin-97 labeled tubules, thus TNFα transport is selectively accomplished by p230-labeled tubules and carriers. This selectivity emulates the same combinations of cargo and golgins on tubules recorded in transfected HeLa cells where TNFα was also seen exclusively in p230 labeled tubules. Thus, TNFα trafficking and secretion in activated macrophages relies preferentially on p230-labeled tubules.

### Example 3

### p230 Tubule Formation is LPS Regulated

Upon activation with LPS, macrophages undergo a dramatic increase in exocytic trafficking activity and, to accomplish that, upregulate the expression of key components of their trafficking machinery. There is a significant increase in the number of GFP-TNFα labeled tubules and carriers emerging from the TGN in LPS activated cells, reflecting the heightened secretory capacity of these cells. The activities of p230GRIP- and golgin-97GRIP-labeled membranes on the TGN were monitored in live cells before and after treatment with LPS. The relative frequency of p230 or golgin97-labeled tubules emerging from the TGN was counted in live activated or resting macrophages. In the absence of LPS, the TGN gives rise to approximately equal numbers of p230 or golgin-97 labeled tubules and carriers (FIG. 2b). However, after LPS activation, the number of golgin-97 tubules/carriers did not change but there was a marked (three fold) increase in the number of p230 tubules emerging from the TGN and an equivalent increase in p230 labeled budding events (FIG. 2b). Thus, there is a selective increase in p230 tubules and carriers accompanying the need to secrete TNF.

To test whether p230 has a functional role in TNFα secretion in macrophages, a vector-based micro RNA (miRNA) system was used to deplete intracellular p230. RAW cells were transfected with the BLOCK-iT (Trade Mark) Pol II miR RNAi Expression Vectors, which contain a GFP reporter gene, and the extent of p230 depletion was determined 48 or 96 hrs after transfection by immunofluorescence. Very little p230 was detected in GFP+ macrophages transfected with miRNA target sequence one (miRNA-1) (FIG. 3a) or two (miRNA-2) whereas control miRNA had no apparent affect on endogenous p230 levels (FIG. 3a). On LPS activation, both control and p230-depleted macrophages showed strong perinuclear staining for TFNα0 (FIG. 3a), demonstrating the production of precursor TNFα in LPS-stimulated macrophages. However, whereas control miRNA macrophages showed high levels of surface TNFα by immunofluorscence and flow cytometry, p230 miRNA transfected macrophages showed very little surface TNFα staining (FIGS. 3b, c). The level of surface TNFα on p230-depleted macrophages was <10% of control macrophages (FIG. 3c). A dramatic reduction of surface TFNα was also observed with a second independent miRNA p230 target as well as an siRNA p230 target thereby ruling out off-target affects of the p230 RNAi. To determine whether all cell surface components were affected by p230 depletion in RAW cells, the surface MHC class II expression was analyzed, which increases following LPS stimulation of macrophages. Surface MHC class II expression was elevated to similar levels in both control and p230-depleted LPS-stimulated macrophages following LPS treatment. Therefore, as for HeLa cells, p230 depletion results in a block of specific cargo from the Golgi apparatus of macrophages. Collectively, these findings demonstrate that p230 is an essential component of the tubules at the TGN required for post-Golgi transport of TNFα.

### Example 4

### Peritoneal Macrophages from Transgenic Mice Expressing p230 miRNA are Blocked in TNFa Secretion

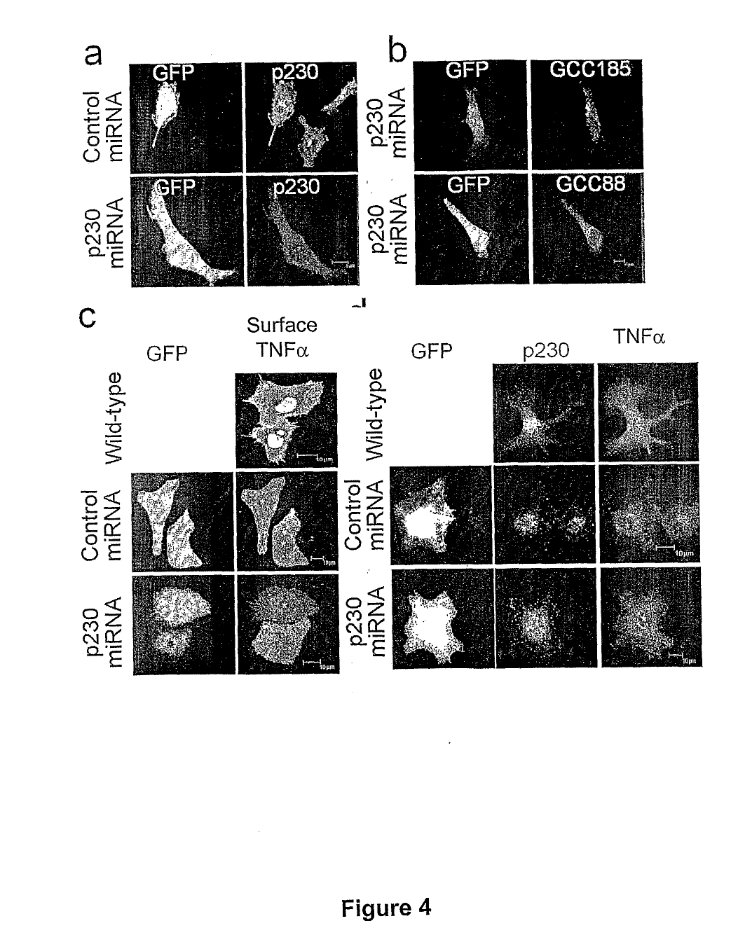

To determine if depletion of p230/golgin-245 could block TNFα secretion in vivo, w transgenic mice were generated expressing RNAi. p230/golgin-245 was silenced in mice by retroviral transduction and bone marrow transplantation. p230 and control miRNA constructs were cloned into a MSCV-based retroviral vector, retrovirus produced and bone marrow cells transduced with the recombinant retrovirus in the presence of IL3, IL6 and SCF. Two days after infection with retrovirus, transduced stem cells were injected into sublethally irradiated recipient mice and peritoneal macrophages were analyzed 8-10 weeks after the transplant. GFP+ macrophages from control and p230 miRNA expressing mice showed the characteristics of wildtype macrophages including extensive membrane ruffling after LPS stimulation. p230miRNA resulted in depletion of p230/golgin-245 in peritoneal macrophages of transgenic mice (FIG. 4a), whereas strong p230 staining was present in control miRNA peritoneal macrophages (FIG. 4a). The staining patterns of other TGN golgins, namely GCC88 and GCC185, were unaffected by the depletion of p230 (FIG. 4b). Following LPS stimulation in the presence of TACE inhibitor, intracellular TNFα was readily detected in both control and p230miRNA macrophages, whereas there was a marked difference in the level of surface TNFα (FIGS. 4c, d). Control miRNA GFP+ macrophages had high levels of surface TNFα whereas there was a considerably lower level of surface TFNα in the miRNA p230 macrophages. Surface TNFα was quantified by confocal microscopy, as described in methods, and GFPbright p230 miRNA macrophages showed no increase in surface fluorescence after LPS treatment compared with resting macrophages, whereas GFPdull macrophages showed an increase in surface fluorescence to ˜25% the level measured in control miRNA macrophages. These findings indicate that the level of expression of the miRNA construct is sufficient to inhibit p230 in vivo, and moreover p230 depletion results in block in post-Golgi transport of TFNα. Furthermore, these analyses demonstrate the applicability of miRNAs to deplete cellular components and disrupt membrane trafficking pathways in vivo.

### Example 5

### Use of miRNA Antagonists

Constructs encoding miRNA directed to the p230/golgin-245 gene are generated and include the receptor GFP as a marker. These are then used to genetically modify stem cells. Enrichment of GFP⁺ stem cells prior to transplantation results in highly efficient reconstitution with ˜80% of thymocytes in transplanted mice GFP⁺. Experiments indicate that p230 miRNA retrogenic mice are resistant to cytokine shock following challenge with LPS, compared with wild-type mice. Groups of two wild-type and p230 miRNA retrogenic mice are administered with 100 mg of LPS intraperitoneally and mice are monitored. Within two hours after treatment, both wild-type mice develop signs of cytokine shock as expected, and showed evidence of distress as assessed by loss of mobility, hunched appearance, and huddling in the corner of the cage. On the other, and the two LPS-treated p230 retrogenic mice, remained healthy and showed no signs of distress two hours after treatment or up to 10 days following LPS treatment. These results show that the silencing of p230 resulted in a physiological reduction in total TNF secretion in the mice. Overall, the studies demonstrate an approach to control cytokine secretion by the specific silencing of p230-mediated trafficking machinery.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Baltimore, J. Virol. 54:401-407, 1985
Bandyopadhyay and Temin, Mol. Cell. Biol. 4:749-754, 1984
Berglund et al, Biotechnology 11:916-920, 1993
Berkner et al, BioTechniques 6:616-629, 1988
Berkner, Curr. Top. Microbiol. Immunol. 158:39-66, 1992
Breakefield and Geller, Mol. Neurobiol. 1:339-371, 1987
Buchschacher and Panganiban, J. Virol. 66:2731-2739, 1982
Erlich et al, J Biol Chem 27104):8328-8337, 1996
Fink et al, Hum. Gene Ther. 3:11-19, 1992
Fink et al, Ann. Rev. Neurosci. 19:265-287, 1996
Freese et al, Biochem. Pharmacol. 40:2189-2199, 1990
Gorziglia and Kapikian, J. Virol. 66:4407-4412, 1992
Helseth et al, J. Virol. 64:2416-2420, 1990
Hodgson, Bio/Technology 9:19-21, 1991
Johnson et al, J. Virol. 66:2952-2965, 1992
Johnson et al, “Peptide Turn Mimetics” in Biotechnology and Pharmacy, Pezzuto et al., Eds., Chapman and Hall, New York, 1993
Lock et al, Traffic 6(12):1142-1156, 2005
Madzak et al, J. Gen. Virol. 73:1533-1536, 1992
Margolskee, Curr. Top., Microbiol. Immunol. 158:67-95, 1992
Miller et al, Mol. Cell. Biol. 5:431-437, 1985
Miller et al, J. Virol. 62:4337-4345, 1988
Miller, Curr. Top. Microbiol. Immunol. 158:1-24, 1992
Moss, Curr. Top. Microbiol. Immunol. 158:25-38, 1992
Moss, Proc. Natl. Acad. Sci. USA 93:11341-11348, 1996
Munder et al, Appl. Microbiol. Biotechnol. 52(3):311-320, 1999
Muzyczka, Curr. Top. Microbiol. Immunol. 158:97429, 1992
Naldini et al, Science 272:263-267, 1996
Nuttal et al, Mol. Immunol. 38:313-316, 2001
Nuttal et al, Eur j Biochem 270:3543-3554, 2003
Ohi et al, Gene 89:279-282, 1990
Page et al, J. Virol. 64:5270-5276, 1990
Petropoulos et al, J. Viol. 66:3391-3397, 1992
Quantin et al, Proc. Natl. Acad. Sci. USA 89:2581-2584, 1992
Remington's Pharmaceutical Sciences, 18^{th} Ed. (1990, Mack Publishing, Company, Easton, Pa., U.S.A.)
Rosenfeld et al, Cell 68:143-155, 1992
Russell and Hirata, Nature Genetics 18:323-328, 1998
Schneider et al, Nature Genetics 18:180-183, 1998
Shimada et al, J. Clin. Invest. 88:1043-1047, 1991
Sorge et al, Mol. Cell. Biol. 4:1730-1737, 1984
Stratford-Perricaudet et al, Hum. Gene Ther. 1:241-256, 1990
Summerton and Weller, Antisense and Nucleic Acid Drug Development 7:187-195, 1997
Young et al, Nat. Biotechnol. 16(10):946-950, 1998
Wilkinson et al, Nucleic Acids Res. 20:2233-2239, 1992

1. A method for controling post-Golgi exocytosis of TNFα, said method comprising introducing to a cell an amount of an antagonist of p230/golgin-245 effective to inhibit the ability of p230/golgin-245 to facilitate exocytosis of TNFα.

2. The method of claim 1 wherein the antagonist inhibits the function, activity, level or operability of p230/golgin-245 or a molecule associated therewith.

3. The method of claim 2 wherein the cell is a eukaryotic cell selected from a macrophage, monocyte, dendritic cell and lymphocyte.

4. The method of claim 3 wherein the eukaryotic cell is a primate cell.

5. The method of claim 4 wherein the primate cell is a human cell.

6. The method of claim 2 wherein the molecule associated with p230/golgin-245 is a G protein.

7. The method of claim 6 wherein the G protein is Arl1.

8. The method of claim 1 in the treatment of an inflammatory disease or condition.

9. The method of claim 8 wherein the inflammatory disease or condition is selected from asthma, chronic obstructive pulmonary disease (COPD), acne, angina, arthritis, aspiration pneumonia disease, colitis, empyema, gastroenteritis, intestinal flu, necrotizing enterocolitis, pelvic inflammatory disease, pharyngitis, pleurisy, raw throat, rubor, sore throat, urinary tract infection and chronic inflammatory demyleinating polyneuropathy and polyradiculoneuropathy.

10. The method of claim 8 wherein the inflammatory disease or condition is acute or chronic inflammation.

11. The method of claim 8 wherein the inflammatory disease or condition is infection by a pathogenic agent.

12. The method of claim 11 wherein the pathogenic agent is Leishmonia.

13. The method of claim 1 wherein the antagonist is an intracellular antibody specific for p230/golgin-245.

14. The method of claim 1 wherein the antagonist is an RNA species which inhibits expression of a gene encoding p230/golgin-245.

15. The method of claim 1 wherein the antagonist is a molecule which crosses cellular membranes.

16. Use of an antagonist of p230/golgin-245 in the manufacture of a medicament in the treatment of an inflammatory condition in a subject.

17. Use of p230/golgin-245 in the manufacture of a medicament in the treatment of an inflammatory condition in a subject.

18. A method for controling post-Golgi exocytosis of TNFα, said method comprising introducing to a cell an amount of an agent which modulates the function, activity, level or operability of p230/golgin-245 or a molecule associated therewith effective to inhibit or promote the ability of p230/golgin-245 to facilitate exocytosis of TNFα.

19. An animal model comprising an elevated level of p230/golgin-245 and which is prone to inflammatory conditions.

20. An animal model comprising a reduced level of p230/golgin-245 and which is resistant to the development of inflammatory conditions.
